(19) United States
(12) Patent Application Publication
(10) Pub. No.: US 2009/0292224 A1
(43) Pub. Date: Nov. 26, 2009

(54) POSITIONING MECHANISM FOR AN INTRODUCER DEVICE

(76) Inventors: Bryan J. Bowman, Indianapolis, IN (US); Donald Duerson, Indianapolis, IN (US)

Correspondence Address: Hologic Cytyc-Suros Division;c/o Rader, Fishman & Grauer, PLLC, 39533 Woodward Avenue, Suite 140, Bloomfield, MI, 48304, US

(21) Appl. No.: 12/469,407

(22) Filed: May 20, 2009

Related U.S. Application Data
(60) Provisional application No. 61/055,325, filed on May 22, 2008.

(51) Int. Cl. A61B 10/02

(52) U.S. Cl. 600/567; 604/272

(57) ABSTRACT

A needle guide is disclosed, including a support member and a body portion. The body portion includes a proximate end that is connected to the support member. The body portion also includes at least one channel extending therethough. The channel is configured for receiving an introducer cannula. At least one latch extends from the support member. A portion of the latch is configured for engagement with a portion of a retaining member.

### CROSS REFERENCE TO RELATED APPLICATIONS

This application claims priority to U.S. provisional application 61/055,325 filed on May 22, 2008, which is incorporated herein by reference in its entirety.

### TECHNICAL FIELD

The present disclosure relates generally to needle guides used for biopsy systems, and in particular to a needle guide configured for positioning an introducer device in a patient.

### BACKGROUND

During certain biopsy procedures, a needle guide may be used through which a portion of an introducer cannula passes. The needle guide serves to minimize non-axial movement or deflection of the introducer during a biopsy to ensure that the introducer is inserted into the correct location of a patient's tissue adjacent a target lesion.

For example, in breast biopsy procedures, such as MRI biopsy procedures, needle guides are often used in connection with a compression plate. The compression plate may include a plurality of grid-like openings. Prior to the start of the breast biopsy procedure, the compression plate is pressed against the breast, thereby compressing it, and a needle guide is inserted into one of the grid-like openings where the lesion to be biopsied may be found. The needle guide may be used through which a portion of an introducer cannula passes. A biopsy needle is then inserted into the introducer.

However, as the biopsy is being taken, the introducer assembly may unintentionally move or shift, particularly in the direction of the z-axis. That is, the introducer cannula may advance further into the breast than intended, or, alternatively, the introducer may partially pull out from the breast as well. One known device for securing the introducer is a spring clip. The spring clip includes an inner diameter that is secured to an outer diameter of the introducer. Thus, the spring clip will restrain the introducer such that the introducer is unable to advance further into the breast. However, the spring clip is unable to restrain the introducer in the opposite direction, and therefore the introducer is still able to partially pull out from the breast.

Accordingly, there exists a need for a device that will secure the introducer such that the introducer is unable to pull out from the body or advance further into the body as a biopsy is being taken.

### SUMMARY

A needle guide is disclosed, including a support member and a body portion. The body portion includes a proximate end that is connected to the support member. The body portion also includes at least one channel extending therethough. The channel is configured for receiving an introducer cannula. At least one latch extends from the support member. A portion of the latch is configured for engagement with a portion of a retaining member.

### BRIEF DESCRIPTION OF THE DRAWINGS

FIG. 1 is an elevational perspective view of a compression plate, a needle guide, a spring clip and a biopsy device including an introducer;

FIG. 2 is an elevational rear perspective view of the needle guide, a portion of the introducer and the spring clip;

FIG. 3 is a front perspective view of the needle guide of FIG. 1;

FIG. 4 is a partially cross-sectional view of the needle guide of FIG. 1;

FIG. 5 is an elevational perspective view of an alternative illustration of the needle guide;

FIG. 6 is an elevational perspective view of another alternative illustration of the needle guide;

FIG. 7 is a top view of the spring clip relaxed; and

FIG. 7A is an enlarged top view of the spring clip in compression.

### DETAILED DESCRIPTION

Referring now to the discussion that follows and also to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present invention. Further, the descriptions set forth herein are not intended to be exhaustive or otherwise limit or restrict the claims to the precise forms and configurations shown in the drawings and disclosed in the following detailed description.

According to various exemplary illustrations described herein, a biopsy system is provided including a needle guide, an introducer and a spring clip. The needle guide includes a support member, a body portion and at least one latch. The body portion of the needle guide includes a proximate end that is connected to the support member. The latch of the needle guide extends from the support member and includes a back surface. The body portion of the needle guide also includes at least one channel extending therethough. The channel is configured for receiving the introducer. The spring clip includes an inner diameter and at least one arm. The inner diameter of the spring clip is in engagement with an outer diameter of the introducer. The back surface of the latch is configured for engagement with a portion the arm of the spring clip. That is, the latch is engaged with the spring clip such that the introducer is unable to move outwardly from the channel.

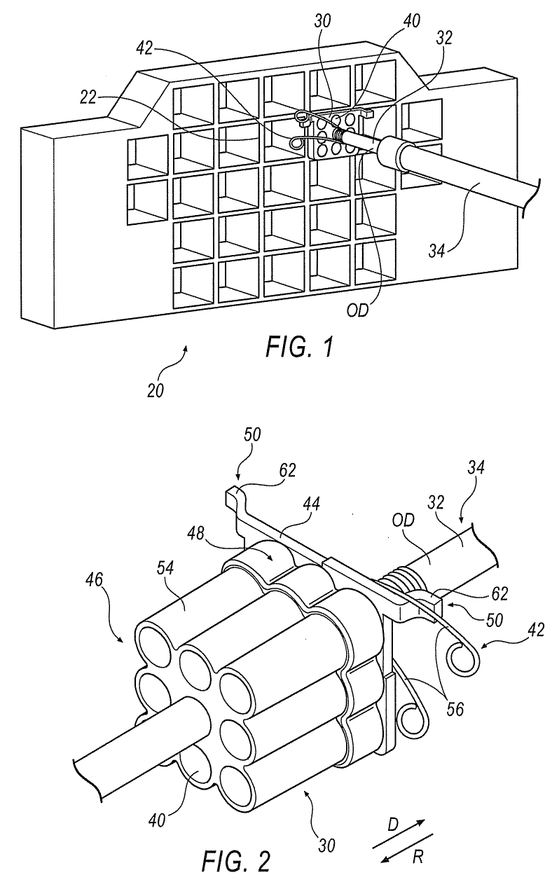

Turning now to the drawings and in particular to FIG. 1, an exemplary compression plate 20 is shown, including a plurality of grid-like openings 22. In the illustration of FIG. 1, the compression plate 20 is used with a needle guide 30. Prior to the start of a biopsy procedure, the compression plate 20 is pressed against the body member to be biopsied. In a breast biopsy procedure, for example, the breast is thereby compressed by the compression plate 20, and an introducer cannula 32 that may be used with, for example, a biopsy device 34, is inserted into one of the openings 40 of the needle guide 30 where the lesion to be biopsied may be found. A spring clip 42 is secured to an outer diameter OD of the introducer 32. The spring clip 42 secures the introducer 32 in place such that the introducer 32 is unable to move inwardly and outwardly from the channel 40 when locked, as discussed in greater detail below.

FIG. 2 is an illustration of the needle guide 30, the spring clip 42 and the introducer 32 taken from the rear or distal end of the needle guide 30. As illustrated, the needle guide 30 includes a support member 44 and a body portion 46. The body portion 46 includes a proximate end 48 that connects the body portion 46 to the support member 44. The support member 44 also includes at least one latch 50. In the illustration as shown, the support member 44 includes two latches 50, located on opposing sides of the support member 44. Although FIG. 2 illustrates the needle guide 30 with two latches 50, only one latch 50, or more than two latches 50 may be used as well. The latch 50 extends from the support member 44 and is configured for engagement with an arm 56 of the spring clip 42, as discussed in greater detail below.

In the illustration shown, the body portion 46 includes nine channels 54 extending therethough. The channels 54 are configured for receiving the introducer cannula 32. In operation, the introducer 32 is slid through one of the channels 54 until the introducer 32 is located in a desired position. The spring clip 42 is then secured to the outer diameter OD of the introducer 32. Although FIG. 2 illustrates the body portion 46 including nine channels 54, it should be noted that any number of channels 54 may be used.

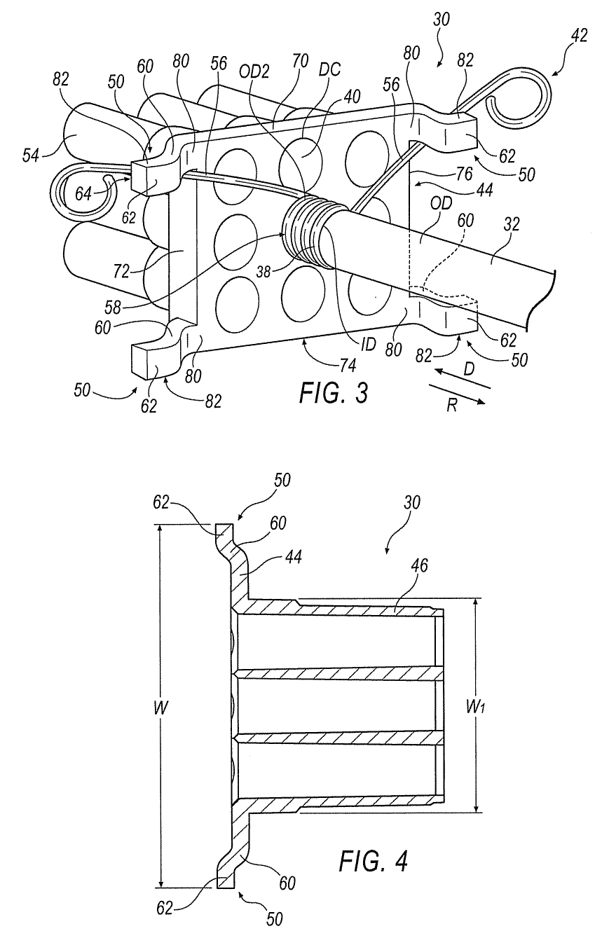

The spring clip 42 retains the introducer 32 to the needle guide 30 such that the introducer 32 is unable to move inwardly and outwardly from the channel 54. As best seen in FIG. 3, an inner diameter ID of the spring clip 42 is in engagement with the outer diameter OD of the introducer 32. An outer coiled portion 58 of the spring clip 42 includes an outer surface 38. The outer surface 38 defines an outer diameter OD2 of the spring clip 42. The outer diameter OD2 is greater than or equal to a diameter DC of the openings 40 of the needle guide 30. Thus, the outer coiled portion 58 retains the introducer 32 such that the introducer 32 is unable to move in the direction D, and further into the channel 54.

Because the latch 50 is engaged with the spring clip 42, the introducer 32 is also unable to move outwardly from the channel 54 when the spring clip 42 is in the locked position. That is, at least one of the arms 56 are in engagement with the latch 50, such that the introducer 32 is unable to move in the direction R and outwardly from the channel 54. Thus, when the spring clip 42 is engaged with the outer diameter OD of the introducer 32 and with the latch 50 of the needle guide 30, the spring clip 42 locks the introducer 32 into the desired position inside of the channel 54 of the needle guide 30.

In one exemplary embodiment, to affect the locking action, the latch 50 includes a raised portion 60 that is curved outwardly towards the direction R. The raised portion 60 allows for the latch 50 to extend outwardly from the channel 54. In the illustration shown in FIGS. 1-4, the latch 50 also includes an outwardly extending tab 62 that is connected to the raised portion 60. The raised portion 60 is located between the support member 44 and the tab 62. A back surface 64 of the latch 50 is in contact with a portion of one of the arms 56 of the spring clip 42, thereby restraining the spring clip 42. Thus, when the latch 50 is engaged with the spring clip 42, the introducer 32 is unable to move outwardly from the channel 54.

One or both of the arms 56 of the spring clip 42 are in engagement with the latch 50 to restrain the introducer 32 in the direction R. Indeed, FIG. 2 illustrates only one of the arms 56 of the spring clip 42 in engagement with the latch 50. Alternatively, FIG. 3 illustrates both of the arms 56 of the spring clip 42 in engagement with the latches 50. FIG. 3 also illustrates the needle guide 30 as including four latches 50. The support member 44 has a generally rectangular profile and includes a top surface 70, a bottom surface 74 and opposing side surfaces 72 and 76. Each of the latches 50 extends from at least one of the surfaces 70, 72, 74 and 76.

In the illustration as shown, each of the latches 50 include a side surface 82 that is aligned with either of the top surface 70 and the bottom surface 74. Although FIG. 3 illustrates the side surface 82 of the latches 50 aligned with the top surface 70 and the bottom surface 74, it should be noted that the side surfaces 82 may also be aligned with the opposing side surfaces 72 and 76. The support member 44 also includes four corners 80, wherein at least one latch 50 extends from one of the four corners 80.

FIG. 4 illustrates the support member 44 including an overall width W that is slightly longer than WI of the body portion 46. Thus, when the needle guide 30 is inserted into one of the openings 22 of the compression plate 20, the support member 44 retains the needle guide 30 in place within one of the openings 22.

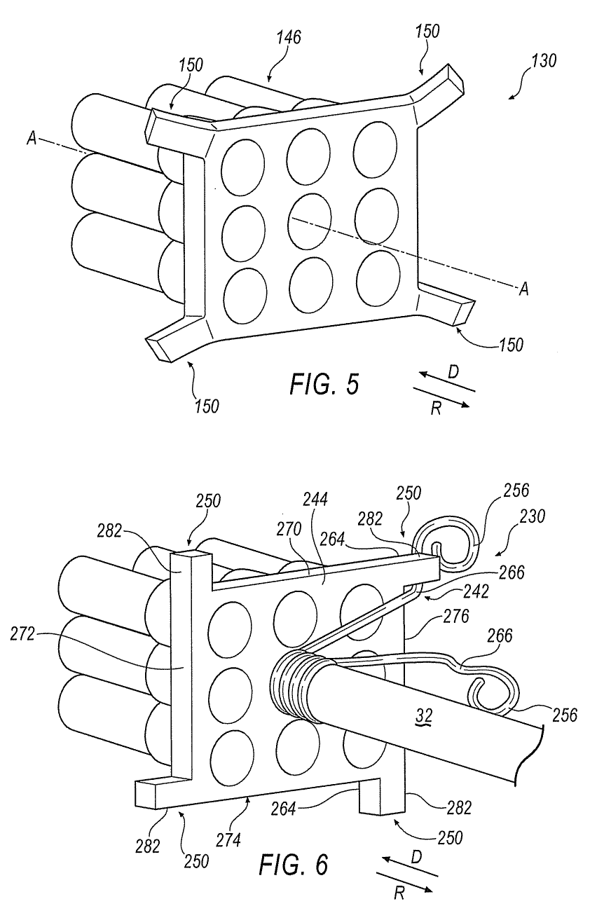

In an alternative illustration as seen in FIG. 5, the needle guide 130 includes four latches 150. Each of the latches 150 extends radially outwardly from a center axis A-A of the needle guide 130. In yet another alternative illustration, as seen in FIG. 6, the needle guide 230 includes four latches 250. Each of the latches 250 include a side surface 282 that is generally aligned with one of the top surface 270, the bottom surface 274, and the opposing side surfaces 272 and 276. It should be noted that while FIGS. 4-6 illustrate the needle guides 130 and 230 including four latches, any number of latches could be used as well.

In the illustration as shown in FIG. 6, the latches 250 do not extend outwardly in the direction R, and away from the channel 254, unlike the latches 50 as seen in FIGS. 1-4. That is, the latches 250 do not include the raised portion 60, but rather are positioned so as to be co-planar with a front face of the support member 244. Thus, the arms 256 of the spring clip 242 may also include a bent portion 266. The bent portion 266 of the arms 256 are configured to extend around the back of the latch 250, and make contact with the back surface 264 of the latch 250. That is, the bent portion 266 wraps around the latch 250, and makes contact with the back surface 264 of the latch 250, thereby inhibiting movement of the introducer 32.

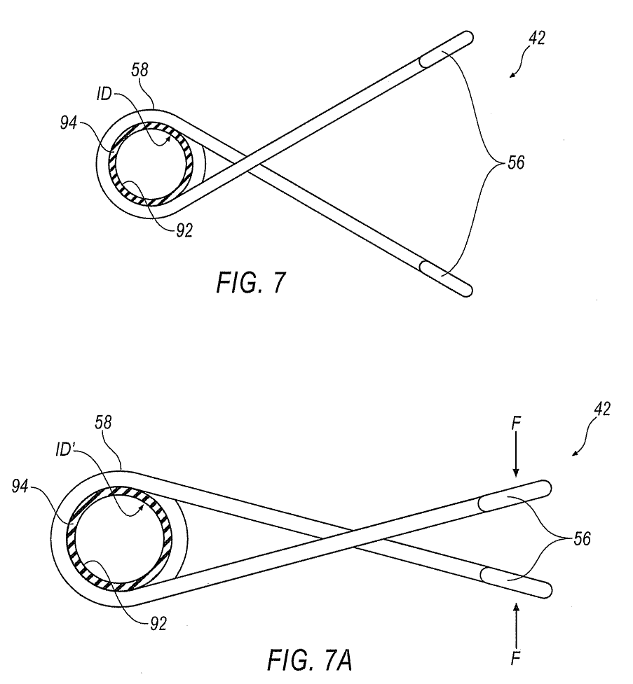

FIG. 7 is a side view of the spring clip 42 when the spring clip is in the relaxed state. FIG. 7A is an enlarged view of the spring clip 42 when the arms 56 are compressed together.

Referring to FIG. 7A, an inner diameter ID′ represents the inner diameter of the outer coiled portion 58 when the arms 56 are compressed together. The arms 56 are compressed together by a manual force F that is selectively applied by a user. When the manual force F is selectively applied, the inner diameter ID′ of the outer coiled portion 58 is greater than the outer diameter OD of the introducer 32. Thus, a user is able to advance the spring clip 42 to a desired position along the outer diameter OD of the introducer 32 by compressing the arms 56 together with the manual force F. This is because the ID′ of the spring clip 42 is greater than the OD of the introducer 32.

However, when the spring clip 42 is relaxed, as seen in FIG. 7, the inner diameter ID of the outer coiled portion 58 is less than an inner diameter ID′. In this configuration, the introducer 32 inhibited from moving the spring clip 42 unless a force is applied to the arms 56.

Moreover, when the manual force F is selectively applied, the arms 56 of the spring clip 42 may be rotated around the outer diameter OD of the introducer 32 by the user as well. That is, because the inner diameter ID′ of the spring clip 42 is greater than the outer diameter OD of the introducer 32, the spring clip 42 is allowed to rotate around the outer diameter OD of the introducer 32.

When the spring clip 42 is situated along the desired position along the outer diameter OD of the introducer 32, the arms 56 are allowed to relax, and the spring clip 42 goes back to the relaxed position as seen in FIG. 7. When in the relaxed state, the inner diameter ID of the outer coiled portion 58 of the spring clip 42 is reduced, thereby engaging the outer diameter OD of the introducer 32. Thus, the spring clip 42 is unable to move along the introducer 32.

The spring clip 42 may be constructed from any flexible material, such as, but not limited to, an MRI compatible material. In the illustrations shown, the inner surface 92 of the outer coiled portion 58 may lined with a material 94. The material 94 is any material that will allow for a friction fit between the spring clip 42 and the outer diameter OD of the introducer 32, such as, but not limited to rubber or polymer.

While the present disclosure has been particularly shown and described with reference to the foregoing preferred embodiments, it should be understood by those skilled in the art that various alternatives to the embodiments of the disclosure described herein may be employed in practicing the disclosure without departing from the spirit and scope of the disclosure as defined in the following claims. It is intended that the following claims define the scope of the disclosure embodiments within the scope of these claims and their equivalents be covered thereby. This description of the disclosure should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A needle guide, comprising:
a support member;
a body portion including a proximate end, wherein the proximate end is connected to the support member; the body portion including at least one channel extending therethough that is configured for receiving an introducer cannula; and
at least one latch extending from the support member;
wherein a portion of the latch is configured for engagement with a portion a retaining member.

2. The needle guide as recited in claim 1, wherein the latch extends outwardly from the support member.

3. The needle guide as recited in claim 2, wherein the latch includes a raised portion, the raised portion located between the support member and a tab member.

4. The needle guide as recited in claim 1, wherein the needle guide includes a plurality of latches.

5. The needle guide as recited in claim 1, wherein the support member is defined by a top surface, a bottom surface, and opposing side surfaces; and wherein the latch extends from one of the surfaces of the support member.

6. The needle guide as recited in claim 5, wherein the latch includes a side surface, the side surface being generally aligned with one of the top surface, the bottom surface, and the opposing side surfaces.

7. The needle guide as recited in claim 1, wherein the support member is defined by a generally rectangular shape having four corners, wherein the latch extends from one of the four corners.

8. The needle guide as recited in claim 1, wherein the latch extends radially outwardly from a center axis of the needle guide.

9. A positioning system for an introducer cannula, comprising:
a needle guide, a retaining mechanism and an introducer cannula;
wherein the needle guide includes a support member, a body portion and at least one latch, wherein the body portion includes at least one channel extending therethough that is configured for receiving the introducer cannula, and wherein the latch extends from the support member;
wherein the introducer cannula is defined by an outer diameter;
wherein the retaining mechanism includes a retaining portion and at least one arm, wherein the retaining portion is defined by an inner diameter that selectively engages with the outer diameter of the introducer cannula;
wherein a portion of the latch is selectively engaged with a portion the arm of the retaining mechanism so as to inhibit movement of the introducer cannula when disposed within the channel.

10. The positioning system as recited in claim 9, wherein the latch extends outwardly from the support member.

11. The positioning system as recited in claim 9, wherein the support member is defined by a top surface, a bottom surface, and opposing side surfaces; and wherein the latch extends from one of the surfaces of the support member.

12. The positioning system as recited in claim 9, wherein the latch includes a side surface, the side surface being generally aligned with one of the top surface, the bottom surface, and the opposing side surfaces.

13. The positioning system as recited in claim 9, wherein the support member is defined by a generally rectangular shape having four corners, wherein the latch extends from one of the four corners.

14. The positioning system as recited in claim 9, wherein the retaining mechanism is a spring clip.

15. The positioning system as recited in claim 14, wherein the spring clip further includes a lining located along the inner diameter for a friction fit between the inner diameter and the outer diameter of the introducer.

16. The positioning system as recited in claim 14, wherein the arm of the spring clip includes a bent portion, the bent portion contacting a surface of the latch.

17. The positioning system as recited in claim 9, wherein the retaining mechanism includes a second arm for engagement with the latch.

18. A method of locking an introducer cannula to a needle guide, comprising:
sliding an introducer cannula through a channel located within a needle guide to a desired position;
securing a spring clip on the introducer, an inner diameter of the spring clip engaged with an outer diameter of the introducer; and
engaging an arm of the spring clip with a latch located on the needle guide such that the latch is engaged with the spring clip so as to inhibit movement of the introducer relative to the support needle guide.

19. The method as recited in claim 18, further comprising the step of rotating the inner diameter of the clip along the outer diameter of the introducer.

20. The method as recited in claim 18, further comprising the step of engaging a second arm of the spring clip with the latch.
